SEQUENCE LISTING (12 sequences)

SEQ ID NO: 1 - DNA, 25 bp, Homo sapiens
  misc_feature (1)..(25): TRAIL forward PCR primer
cagaggaagaagcaacacattctct

SEQ ID NO: 2 - DNA, 25 bp, Homo sapiens
  misc_feature (1)..(25): TRAIL reverse PCR primer
tgatgattcccaggagtttattttg

SEQ ID NO: 3 - DNA, 27 bp, Homo sapiens
  misc_feature (1)..(27): TRAIL fluorogenic probe
actccaagaatgaaaaggctctgggcc

SEQ ID NO: 4 - DNA, 22 bp, Homo sapiens
  misc_feature (1)..(22): CD95L forward PCR primer
atgcacacagcatcatctttgg

SEQ ID NO: 5 - DNA, 20 bp, Homo sapiens
  misc_feature (1)..(20): CD95L reverse PCR primer
atgggccactttcctcagct

SEQ ID NO: 6 - DNA, 26 bp, Homo sapiens
  misc_feature (1)..(26): CD95L fluorogenic probe
aagcaaataggccaccccagtccacc

SEQ ID NO: 7 - DNA, 21 bp, Homo sapiens
  misc_feature (1)..(21): MxA forward PCR primer
cagcacctgatggcctatcac

SEQ ID NO: 8 - DNA, 24 bp, Homo sapiens
  misc_feature (1)..(24): MxA reverse PCR primer
gagcatgaagaactggatgatcaa

SEQ ID NO: 9 - DNA, 24 bp, Homo sapiens
  misc_feature (1)..(24): MxA probe fluorogenic probe
agcaagcgcatctccagccacatc

SEQ ID NO: 10 - DNA, 25 bp, Homo sapiens
  misc_feature (1)..(25): HPRT forward PCR primer
agtctggcttatatccaacacttcg

SEQ ID NO: 11 - DNA, 22 bp, Homo sapiens
  misc_feature (1)..(22): HPRT reverse PCR primer
gactttgctttccttggtcagg

SEQ ID NO: 12 - DNA, 26 bp, Homo sapiens
  misc_feature (1)..(26): HPRT fluorogenic probe
tttcaccagcaagcttgcgaccttga

(19) United States
(12) Patent Application Publication
(10) Pub. No.: US 2007/0077557 A1
(43) Pub. Date: Apr. 5, 2007

(54) Method for identifying type-1 interferon responsive ms patients by determining trail expression

(76) Inventors: Klaus-Peter Wandinger, Berlin (DE); Jan Lunemann, Berlin (DE); Frauke Zipp, Berlin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/513,312

(22) PCT Filed: May 29, 2003
(86) PCT No.: PCT/EP03/05650

§ 371 Date: Apr. 11, 2006

(30) Foreign Application Priority Data
May 29, 2002 (EP) 02011964.0

(51) Int. Cl. C12Q 1/68; G01N 33/567

(52) U.S. Cl. 435/6; 435/7.2

(57) ABSTRACT

This invention relates to a method for identifying MS patients susceptible to type I-Interferon (IFN), in particular IFN-β, and for monitoring IFN therapy in MS patients by determining TRAIL gene and/or protein expression as a marker.

This invention relates to a method for identifying MS patients susceptible to type I-Interferon (IFN), in particular IFN-β, and for monitoring IFN therapy in MS patients by determining TRAIL gene and/or protein expression as a marker.

Multiple Sclerosis (MS) affects approximately 1 million individuals world-wide and is the most common disease of the central nervous system (CNS) that causes prolonged and severe disability in young adults. Although its aetiology remains elusive, strong evidence supports the concept that a T cell-mediated inflammatory process against self-molecules within the white matter of the brain and spinal cord underlies its pathogenesis (Martin et al. 1992, Annu Rev Immunol 10: 153-187). Since myelin-reactive T cells are present in both MS patients and healthy individuals (Pette et al. 1990, Neurology 40: 1770-1776; Hohlfeld et al. 1995, Neurology 45 (suppl 6): S33-S38), the primary immune abnormality in MS most likely involves failed regulatory mechanisms that lead to an enhanced T cell activation status and less stringent activation requirements (Zang et al. 1994, J Exp Med 179: 973-984; Markovic-Plese et al. 2001, J Clin Invest 108: 1185-1194).

Interferon-beta (IFN-β), a type-I Interferon (IFN), is a pleiotropic cytokine with immunomodulatory properties and has become a global standard in the treatment of MS. Despite the well documented efficacy in responders to this medication, a substantial number of patients fail to respond to IFN-β. Why IFN-β therapy is or is not effective with respect to MS, and how IFN-β alters the clinical course of MS remains unclear (The IFNB Multiple Sclerosis Study Group and the University of British Columbia MS/MRI Analysis Group 1995, Neurology 45: 1277-1285; Jacobs et al. 1996, Ann Neurol 39: 285-294; PRISMS Study Group 1998, Lancet 352: 1498-1504). Putative mechanisms of action include the inhibition of T cell proliferation, regulation of a large number of cytokines, and blocking of blood-brain barrier opening via interference with cell adhesion, migration and matrix metalloproteinase activity (Rudick et al 1996, Ann Neurol 40: 618-627; Wang et al. 2000, J Immunol 165: 548-557; Stone et al. 2000, Ann Neurol 37: 611-619). Understanding how IFN-β works in MS, however, is imperative for a development of markers that would allow predicting an individual's chance of a positive IFN-β response. In the face of the costs of IFN-β therapy as well as possible side-effects for the patient, such a prediction might be valuable in increasing the efficacy of current and future strategies to treat patients suffering from MS.

Consequently, there is a need for identifying markers which correlate with the clinical outcome of IFN-β therapy in MS patients, and which are usable in a method for predicting therapy response of an individual patient.

Recently, a number of genes involved in innate and specific immune responses that are potential effector targets of IFN-β in MS were identified (Wandinger et al 2001, Ann Neurol 50: 349-357). Tumour necrosis factor (TNF)-related apoptosis inducing ligand (TRAIL; also referred to as APO2L), a novel member of the TNF/nerve growth factor superfamily (Wiley et al. 1995, Immunity 3: 673-682), was among the genes that were consistently up-regulated by IFN-β in vitro. Interestingly, TRAIL has also been shown to mediate the induction of endogenous IFN-β, thereby amplifying the effects of this therapy (Kumar-Sinha et al. 2002, J Biol Chem 277: 575-585).

Apart from its apoptosis inducing effects (Wiley et al. 1995, Immunity 3: 673-682), TRAIL was recently shown to exert potent anti-inflammatory properties at the T cell level (Song et al. 2000, J Exp Med 191: 1095-1104; Hilliard et al. 2001, J Immunol 166: 1314-1319; Lünemann et al. 2002, J Immunol 168: 4881-4888). Studies on animal models of autoimmune diseases demonstrated that systemic neutralisation by TRAIL receptor 2 led to exacerbation of collagen-induced arthritis and experimental autoimmune encephalomyelitis and suggested an influence of TRAIL on T cell growth and effector function (Song et al. 2000, J Exp Med 191: 1095-1104; Hilliard et al. 2001, J Immunol 166: 1314-1319). In the human immune system, Lünemann et al. (2002, J Immunol 168: 4881-4888) have very recently shown that TRAIL fails to induce apoptosis, but inhibits the activation of autoreactive and foreign antigen-specific T cells. In particular, TRAIL negatively regulates calcium influx through store-operated calcium release-activated calcium channels which is crucial to lymphocyte activation, and moreover inhibits subsequent cell cycle progression as well as cytokine production independently of the T cell's antigen-specificity or the T helper phenotype (Lünemann et al. 2002, J Immunol 168: 4881-4888). This impact of TRAIL on human T cell effector functions suggests a role of the TRAIL system in the pathogenesis of MS in which activated myelin-specific T cells are thought to mediate the inflammatory brain damage. This notion is supported by the fact that TRAIL was found to be elevated in peripheral immune cells of MS patients during the natural disease course, and that levels were highest in patients with relapsing-remitting MS at the time of clinical remission (Huang et al. 2000, Neurology 55: 928-934).

In addition to its immunoregulatory properties on human T cells, TRAIL was shown to induce apoptotic cell death in nontransformed human hepatocytes and brain cells (Jo et al. 2000, Nat Med 6: 564-657; Nitsch et al. 2000, Lancet 356: 827-828). The latter finding suggests a two edged role for TRAIL in neuroinflammation by promoting both anti-inflammatory and destructive properties. However, given the immediate effects of IFN-β on the integrity of the blood-brain barrier, the IFN-β-induced TRAIL up-regulation in the treatment of MS more likely comprises immunoregulatory mechanisms in the periphery than direct effect on the CNS (Stone et al. 1995, Ann Neurol 37: 611-619).

Up till now it has not been possible to predict a patient's susceptibility to IFN-β treatment with a high level of confidence. Given that the IFN-β treatment is extremely expensive and places an immense financial burden on the health system, it would be desirable to be able to make predictions about the outcome of an IFN-β therapy to a patient. Moreover, for the individual patient it is highly desirable to have such a prediction in order to weigh up the probability of benefit on the one hand and side-effects that have to be expected on the other.

Accordingly, it has been an object of the present invention to be able to predict the chances of a successful IFN-β therapy in MS affected individuals.

This object is solved by a method for identifying an individual as a positive responder or a negative responder, i.e. non-responder, to type-I IFN therapy used in the treatment of MS, characterized in that TRAIL expression and/or TRAIL protein level is determined.

It is also solved by the use of the method according to the present invention for monitoring type-I IFN therapy used in the treatment of MS.

It is also solved by the use of the method according to the present invention for monitoring the response of a MS patient to higher IFN-β doses.

It is also solved by the use of the method according to the present invention for monitoring the development and/or occurrence of neutralising antibodies (NAB) in the course of IFN-β treatment.

It is furthermore solved by a kit for performing the method according to the present invention, comprising a nucleic acid probe and/or one or more primers specific for the TRAIL gene or a portion thereof and/or an antibody specific for the TRAIL polypeptide or a portion thereof. In another preferred embodiment, the kit (or pharmaceutical composition) for performing the method according to the present invention comprises a standardised TRAIL protein probe (i.e. blood serum or plasma etc.) and one or more anti-TRAIL antibodies, optionally together with other suitable components, either in admixture with another and/or or in separate containers. Such kits can be suitably used for, amongst others, ELISA assays, preferably in large scale automated proceedings.

Preferably, the method of the present invention is used in an IFN-β therapy.

The method of the present invention is preferably used to determine TRAIL expression and/or TRAIL protein level prior to IFN therapy.

In said method, a sample is preferably obtained from a mammalian, and more preferably is obtained from a human.

In a preferred embodiment, the sample is selected from the group comprising a bodily fluid, a fraction thereof, tissue extract, and cell extract.

In a more preferred embodiment, the bodily fluid is selected from the group comprising whole blood, blood serum, plasma, cerebrospinal fluid, synovial fluid, ascites exudate, inflammatory exudate, and urine.

In a particular preferred embodiment of the method according to the present invention,
(a) TRAIL expression is determined by detecting and/or measuring the amount and/or concentration of TRAIL nucleic acids encoding the TRAIL polypeptide or parts thereof, and
(b) TRAIL protein level is determined by detecting and/or measuring the amount and/or concentration of TRAIL polypeptide or parts thereof.

In one embodiment of the method according to the present invention, TRAIL protein level determination is performed by measuring the amount and/or concentration of soluble TRAIL (sTRAIL) protein in a liquid sample.

The measurement of the amount and/or concentration of TRAIL polypeptide is preferably done by an immunoassay using an anti-TRAIL antibody.

The immunoassay preferably is an enzyme-linked immunosorbent assay (ELISA) but also may be, for example, a radioimmunoassay (RIA) or a blotting technique such as Western blot.

The amount and/or concentration of TRAIL polypeptide determined preferably is compared to a limiting value discriminating positive responders and non-responders, which limiting value is about 350±20 pg/ml. Nevertheless, also higher levels of sTRAIL of about 584.1 pg/ml prior to therapy correctly predicted the clinical outcome in 90.5% of the responders and 71.4% of the non-responders.

Alternatively, the level of membrane bound TRAIL protein may be determined, which is preferably done by using an anti-TRAIL antibody in, for example, FACScan analysis, ELIs-pot assay or an immunhistochemical technique.

In another embodiment of the present invention, the amount and/or concentration of TRAIL nucleic acid is determined by preferably measuring the amount and/or concentration of TRAIL mRNA in a sample of tissue or cell extract.

In a preferred embodiment, said sample comprises an extract selected from the group comprising an extract of blood cells, peripheral immune cells, neuronal tissue and muscle tissue, of which an extract of peripheral blood mononuclear cells (PBMC) is particularly preferred.

Measurement of the amount and/or concentration of TRAIL mRNA is preferably done with the aid of the polymerase chain reaction (PCR) using one or more TRAIL specific probes.

Alternatively, other techniques for determining nucleic acids such as Northern blot may be used. Furthermore, the amount and/or concentration of TRAIL mRNA may be determined in solid tissue or cell specimens by, for example, in situ hybridisation using TRAIL specific probes.

The amount and/or concentration of TRAIL mRNA preferably is compared to a limiting value discriminating positive responders and non-responders. Said limiting value is at least twofold compared to the baseline.

The amount and/or concentration of TRAIL nucleic acid and/or polypeptide is preferably correlated with the amount and/or concentration of a nucleic acid and/or polypeptide, respectively, of one or more biological response markers of IFN-β therapy, such as M×A protein or any other IFN-inducible protein.

As used herein, an individual suffering from manifested MS is diagnosed according to generally accepted criteria (Poser et al. 1983, Ann Neurol 13: 227-231; McDonald et al. 2001, Ann Neurol 50: 121-127). Disability status (as defined by the Expanded Disability Status Scale, EDSS; Kurtzke 1983; Neurology 22: 1444-1452) was assessed by clinical parameters.

As used herein, “positive responders” are defined as patients who experienced no further relapses and no deterioration in the EDSS during IFN-β treatment, whereas patients who continue to have one or more relapses are defined as “non-responders”. In another aspect, positive responders and non-responders to IFN-β treatment are defined by paraclinical activity markers, e.g. magnetic resonance imaging (MRI).

The method of the present invention shall be understood as a method wherein determination of TRAIL expression and/or TRAIL protein level is carried out extracorporally.

The term “determination” as used herein may mean both qualitatively detecting and quantifying.

As used herein, an “anti-TRAIL antibody” is an antibody directed against TRAIL polypeptide or parts thereof and may comprise polyclonal antiserum, monoclonal antibodies, diabodies and chimera.

As used herein, “TRAIL” relates to the tumour necrosis factor (TNF)-related apoptosis-inducing ligand, or “TRAIL” for short, is a molecule first noted for inducing apoptosis, a certain type of cell death with specific morphological features. Both TNF and TRAIL belong to the same family of molecules with similar capacities. Since its initial discovery, other functions of the TRAIL molecule have been identified. The function important for the data presented here is an inhibition of T cell actions, a function which can be considered anti-inflammatory because of the overall prevention of inflammation. TRAIL produces this effect by inhibiting certain pathways of the cell cycle responsible for the increase in the number of T cells (proliferation, see next item).

As used herein “T cell proliferation/cell cycle” relates to an increase in the number of T cells is called T cell proliferation. Prerequisite for this process are a series of events starting with an activation phase in which Calcium plays a major role, and leading subsequently to division of a single cell into two cells. For the latter to take place, cells have to go through a so-called “cell cycle” which consists of several steps (e.g. G (=gap) phases and an S (=synthesis) phase) and which is regulated by several molecules.

“Gene expression profiling” provides a snapshot of the genes expressed in specific tissues at specific points in time by means of, for example, cDNA microarrays, i.e. microarrays containing hundreds and thousands of different probes for simultaneous measurement of gene expression.

As used herein, “Real-time quantitative rtPCR” relates to a method that monitors the degradation of a dual-labelled fluorescent probe in real time concomitant with PCR amplification. Input target RNA levels are correlated with the time (measured in PCR cycles) at which the reporter fluorescent emission increases beyond a threshold level.

As used herein “ROC analysis” relates to a statistical method to quantify how accurately a diagnostic test performs when it is required to make a series of discriminations into two different states (diseased and non-diseased) on the basis of a certain diagnostic parameter. Every value of that discriminating parameter is used as a cut-off with calculation of the corresponding sensitivity and specificity.

In summary, TRAIL is disclosed herein as the first biological response marker of IFN-β therapy in MS that has been identified. It is demonstrated that early and sustained TRAIL gene induction is a marker of IFN-β therapy response by linking its gene and protein expression profile with clinical disease markers in long-term treated MS patients. More detailed, non-responders to IFN-β therapy can be distinguished from responsive individuals by their in vivo profile of TRAIL gene and protein expression. Furthermore, elevated sTRAIL protein levels in patient sera allowed to predict treatment response even before therapy was initiated. Using an ELISA for determining sTRAIL serum concentration, two limiting values, one of about 350±20 pg/ml and a higher one, namely 584.1 pg/ml were observed. Thus, concentrations above these limiting values indicate a prediction of positive response to IFN-β therapy, whereas lower concentrations point to a response failure.

The invention shall now be further described by the following examples with respect to the attached figures. All examples are provided by way of example only, without any intended limitation of the scope of the invention. All cited references are incorporated herein by reference in their entireties.

Prior to outlining the examples, reference is made to the figures, wherein

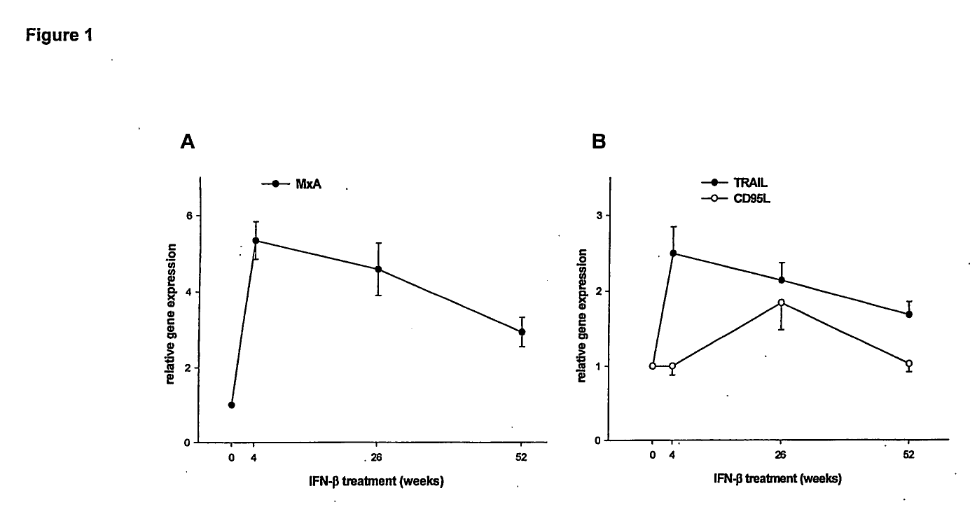

FIG. 1 shows the spontaneous gene expression of M×A, TRAIL and CD95L in 62 MS patients during IFN-β-1a therapy. Samples of peripheral blood mononuclear cells (PBMC) were obtained before the beginning of IFN-β-1a therapy and at weeks 4, 26 and 52 under therapy. Total RNA was extracted and relative mRNA levels were quantified by realtime (rt)-PCR. Gene expression levels were normalised to expression of hypoxanthine phosphoryltransferase (HPRT). Induction of M×A protein gene expression was measured as a biological response marker to IFN-β therapy in vivo. Changes are expressed relative to gene expression levels before treatment. Data are presented as mean±SEM. Time influence proved by one-group MANOVA, each p<0.05. Post-hoc Wilcoxon tests of changes after 4, 26 and 52 weeks of treatment compared to baseline multiple adjusted (*p<0.001).

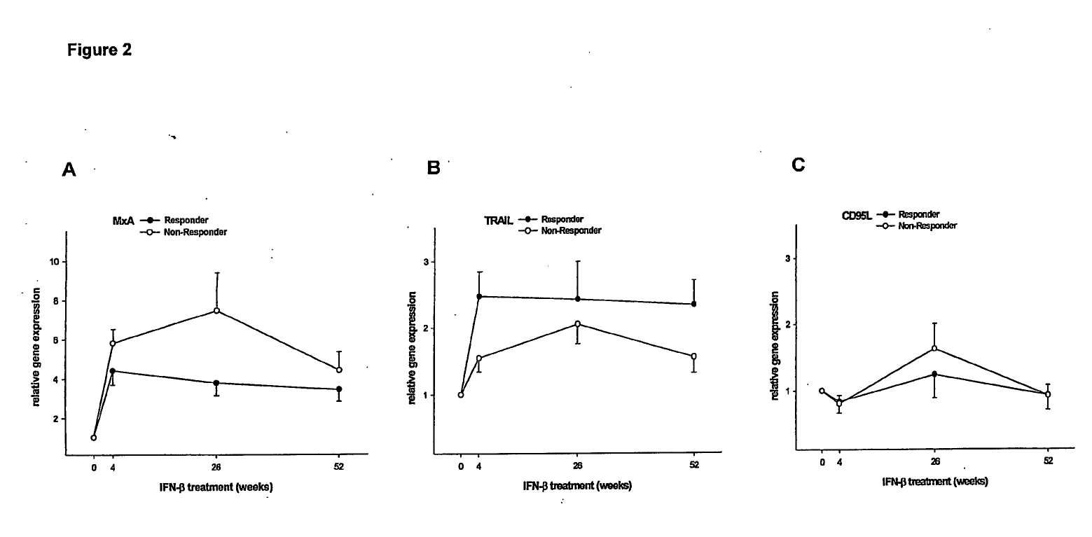

FIG. 2 shows the differential induction of TRAIL gene expression in IFN-β ‘first year responders’ (n=20) and ‘first year non-responders’ (n=19). In contrast to non-responders, IFN-β-1a therapy resulted in early and sustained up-regulation of TRAIL gene expression in MS patients with clinical treatment response. Up-regulation of M×A gene expression demonstrates the in vivo biological effectiveness of IFN-β-1a in both patient groups. CD95L was not significantly regulated by IFN-β therapy in any patient subset. *p<0.05.

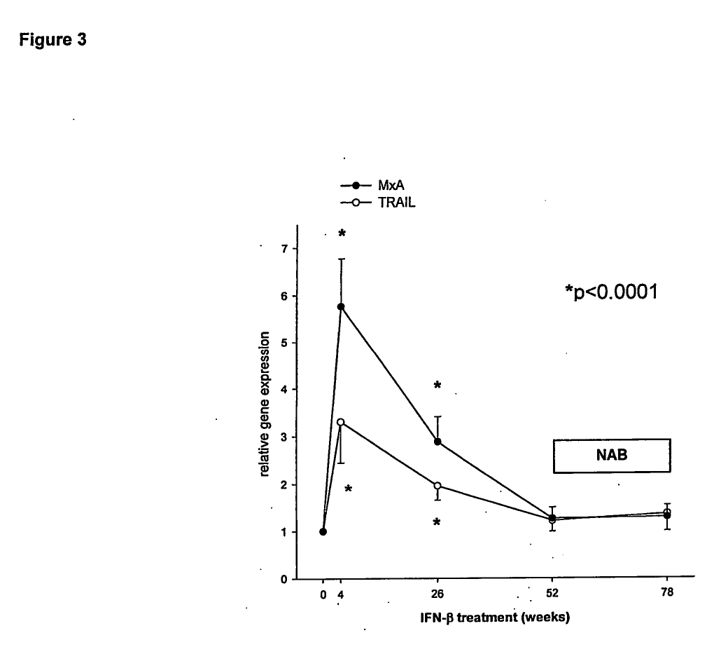

FIG. 3 shows that the up-regulation of TRAIL gene expression during IFN-β treatment is abrogated in the presence of neutralising antibodies (NAB). Spontaneous gene expression of M×A and TRAIL was measured in a third subset of patients (n=23) who developed NAB to IFN-β-1a after 1 year of treatment. Presence of NAB was confirmed after 18 months. Initial up-regulation of M×A and TRAIL gene expression was abrogated in the presence of neutralising antibodies. Time effect: one-group MANOVA, each p<0.0001, post-hoc Wilcoxon tests multiple adjusted (*p<0.001).

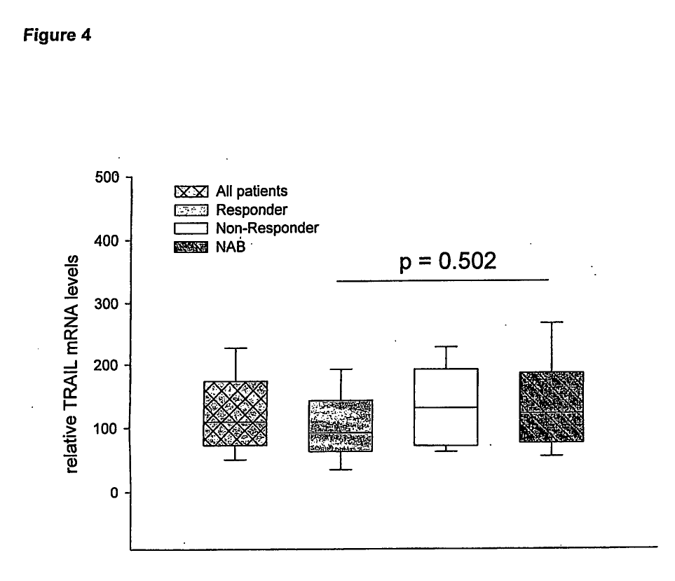

FIG. 4 shows baseline TRAIL gene expression levels in patient subsets. Gene expression levels were normalised to expression of HPRT. Box-whisker plots depict the median level for each group (horizontal line in the box), the 25^{th} and 75^{th} percentiles (upper and lower edges of box), and the 10^{th} and 90^{th} percentiles (lines extending above and below the box). No significant differences were observed between the patient subgroups (Mann-Whitney-U test).

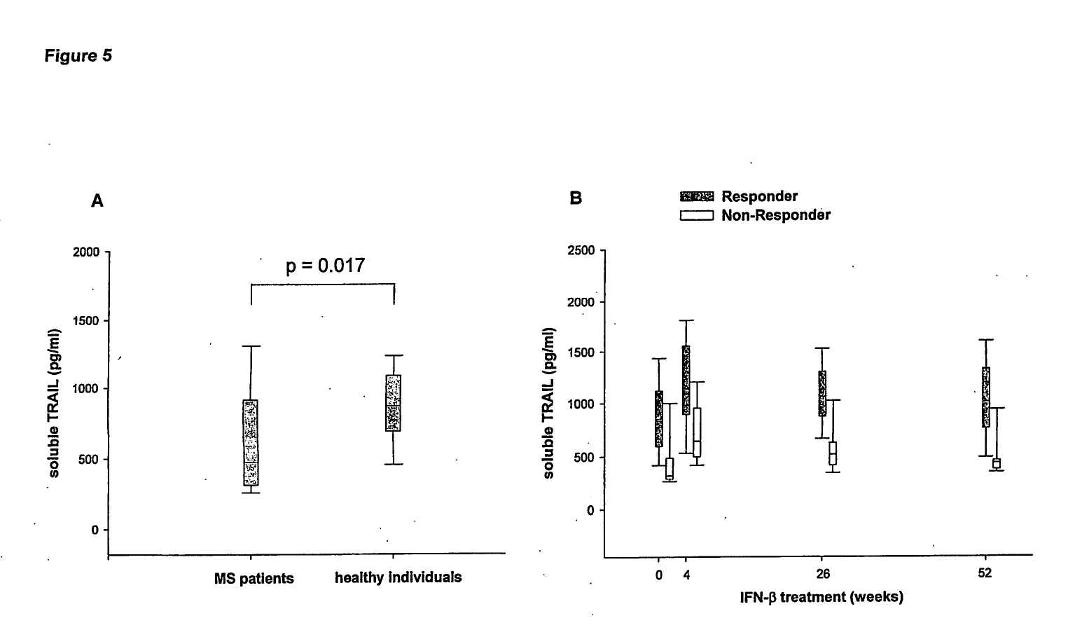

FIG. 5 shows soluble TRAIL protein levels in MS patients compared to healthy individuals (A) and during IFN-β-1a therapy (B). For experimental details, see example 4.

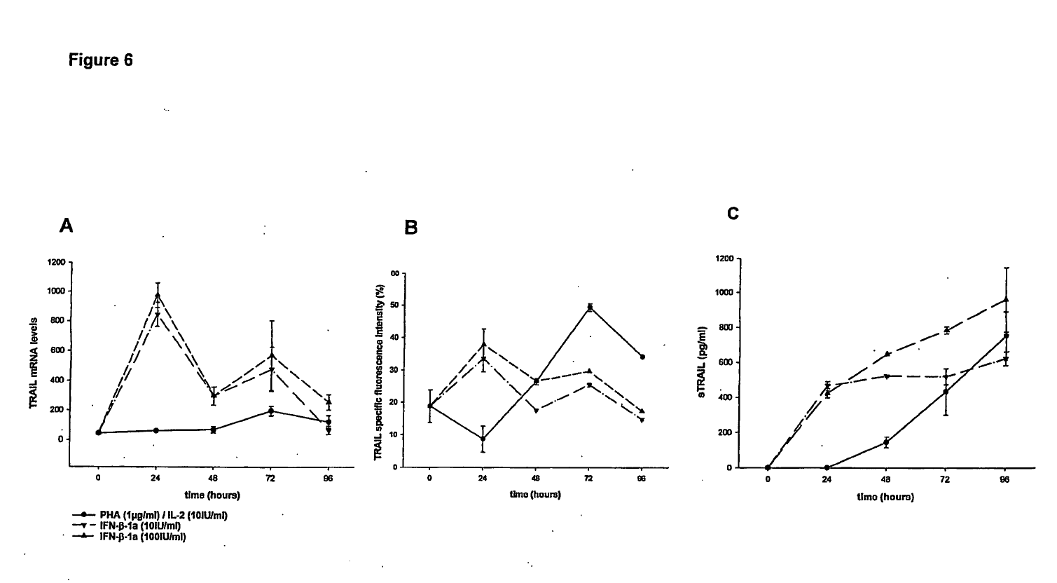

FIG. 6 shows the regulation of TRAIL gene transcription and protein expression by IFN-β. PBMC (10⁶ per well) were cultured for the indicated time periods in 48 well plates in a final volume of 1 ml. Cells were incubated with IFN-β-1a (10 and 100 IU/ml) of PHA (1 μg/ml) and Interleukin-2 (IL-2) (10 IU/ml). TRAIL mRNA was quantified by rtPCR. Gene expression levels were normalised to 18S rRNA (A). Cell surface expression was detected by flow cytometry. Cells were stained with an anti-TRAIL monoclonal antibody, followed by phycoerythrin (PE)-labelled goat anti-mouse monoclonal antibody (B). Concentration of sTRAIL protein in culture supernates was determined by ELISA (C). Results obtained in 2 healthy individuals are shown (mean±SEM).

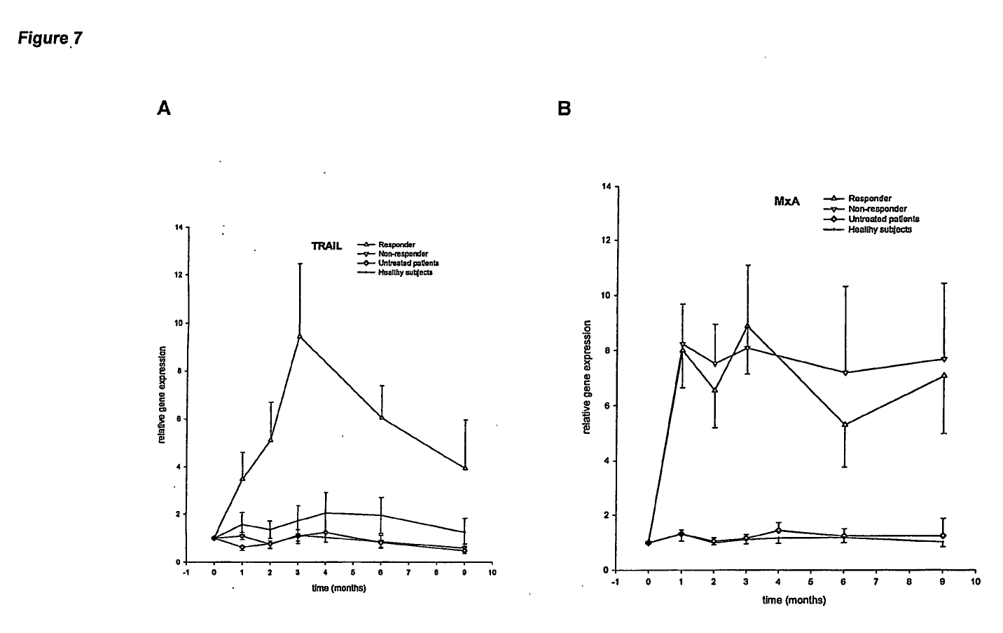

FIG. 7 shows that TRAIL gene expression is specifically induced in IFN-β treated patients responsive to therapy. Spontaneous gene expression was measured in a second, independent cohort of treated and untreated patients as well as healthy subjects over a period of 9 months. For demographic characteristics of patients see table 1b. A significant influence on TRAIL gene expression was exclusively seen in IFN-β treatment responders. (MANOVA p=0.026) (A). Upregulation of M×A gene expression demonstrates the in vivo biological effectiveness of IFN-β-1a in treatment responders (MANOVA p=0.0064) and non responders (MANOVA p=0.0006) (B).

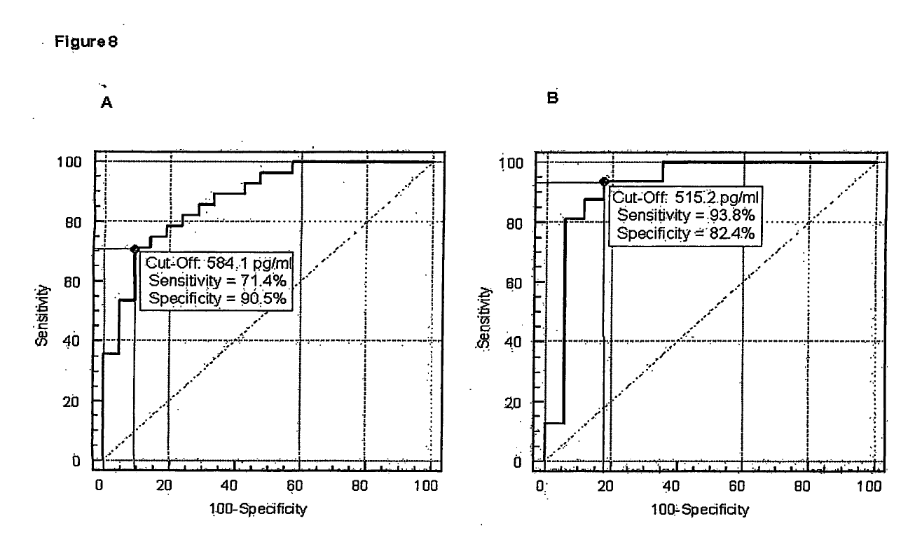

FIG. 8 shows the predictive discrimination of sTRAIL levels in ‘first year responders’ versus ‘first year non-responders’. Sera from 29 responders and 20 non-responders (including patients with NAB) prior to treatment were investigated (A). ROC-characteristics showing sensitivity and specificity for predicting treatment response by sTRAIL levels in patients' sera prior to IFN-β therapy (see Materials and Methods). Area under the ROC curve=0.879, 95%-confidence interval: 0.785-0.974. (B) ROC-analysis in a subgroup of patients without NAB (16 responders and 17 non-responders). Area under the ROC curve=0.919, 95%-confidence interval: 0.812-1.026.

TABLE 1a
Clinical characteristics of patients treated with IFN-β-1a 44 μg once weekly

| | | | | Duration of | | | | | NAB | NAB |
|---|---|---|---|---|---|---|---|---|---|---|
| | | | | definite | | EDSS | Relapses | Relapses | titers | titers |
| | | | | MS | EDSS | week | two years | during study | (INU/ml) | (INU/ml) |
| | | Mean | | (months) | baseline | 52/78 | prior to study | period | week 52 | week 78 |
| Patient | | age, y | Sex, | Mean | median | Median | mean | mean | Mean | mean |
| groups | N (%) | (range) | F/M | (range) | (range) | (range) | (range) | (range) | (range) | (range) |
| Total | 62 (100) | 33.8 | 45/17 | 68.9 | 2.3 | 2.3 | 2.9 | 0.8 | 286.8 | 376.6 |
| | | (22-44) | | (1-307) | (0.0-5.5) | (0.0-6.5) | (2-10) | (0-4) | (0-2001) | (0-2001) |
| R | 20 (32) | 35.0 | 12/8 | 51.52 | 2.2 | 1.8 | 2.6 | 0 | 0 | 0 |
| | | (25-64) | | (7-150) | (1.0-3.5) | (1.0-3.5) | (2-6) | | | |
| NR | 19 (31) | 34.8 | 15/4 | 61.2 | 2.6 | 3.0 | 2.9 | 1.9 | 0 | 0 |
| | | (27-39) | | (1-216) | (1.0-5.5) | (1.0-6.5) | (2-5) | (1-3) | | |
| NAB | 23 (37) | 33.0 | 18/5 | 90.4 | 2.3 | 2.1 | 3.0 | 0.6 | 773.1 | 1111.1 |
| | | (22-44) | | (4-307) | (0.0-4.0) | (1.0-5.0) | (2-10) | (0-4) | (100-2001) | (230-2001) |

R: ‘first year responder’;
NR: ‘first year non-responder’;
INU: Interferon neutralising units;
NAB: Neutralising antibodies. Presence of NAB was confirmed after 18 months.

TABLE 1b
Clinical and MRI characteristics of untreated patients and patients treated with IFN-β-1a 22 μg three times/week

| | | | | | | | | | T2 lesion | T1 lesion | New Gd+ |
|---|---|---|---|---|---|---|---|---|---|---|---|
| | | | | Duration | | | Relapses | Relapses | load | load | lesions |
| | | | | of definite | | EDSS | two years | during | baseline/ | baseline/ | during |
| | | Mean | | MS | EDSS | end of | prior to | study | end of | end of | study |
| | | age | | (months) | baseline | study | study | period | study | study | period |
| Patient | | (years) | Sex | Mean | Median | median | Mean | mean | mean | mean | mean |
| groups | n (%) | (range) | F/M | (range) | (range) | (range) | (range) | (range) | (range) | (range) | (range) |
| Total | 20 (100) | 33.3 | 17/3 | 34.5 | 1.5 | 1.4 | 1.45 | 0.45 | 19.7/20.9 | 6.7/8.4 | 1.8 |
| | | (18-47) | | (1-196) | (0.0-4.0) | (0-4) | (1-3) | (0-2) | (2-83)/ | (0-24)/ | (0-9) |
| | | | | | | | | | (5-63) | (1-23) | |
| R | 5 (25) | 34.2 | 2/3 | 43 | 1.6 | 1.2 | 1.4 | 0.2 | 30.6/25.0 | 8.6/8.0 | 0 |
| | | (27-40) | | (3-123) | (1.0-2.0) | (1.0-2.0) | (1-3) | (0-1) | (5-83)/ | (2-20)/ | (0-0) |
| | | | | | | | | | (5-63) | (2-18) | |
| NR | 6 (30) | 32.7 | 6/0 | 62 | 2.0 | 2.0 | 1.83 | 0.83 | 23.7/28.0 | 9.7/12.5 | 4.0 |
| | | (18-47) | | (9-196) | (0.0-4.0) | (1.0-4.0) | (1-3) | (0-2) | (4-45)/ | (4-21)/ | (1-7) |
| | | | | | | | | | (17-45) | (7-23) | |
| Untreated | 9 (45) | 33.2 | 9/0 | 11 | 1.1 | 1.1 | 1.22 | 0.33 | 10/13 | 4.8/5.5 | 0.5 |
| | | (21-46) | | (1-34) | (0.0-3.0) | (0.0-2.0) | (1-3) | (0-2) | (2-28)/ | (0-24)/ | (0-2) |
| | | | | | | | | | (5-34) | (1-21) | |

R: First year responder;
NR: First year non-responder

### EXAMPLES

The following examples are intended to illustrate and to substantiate the present invention. In order to study an involvement of TRAIL in IFN-β therapy, patients with manifested MS were recruited. The study was approved by the local ethical committee, and informed consent was obtained from each individual.

All MS patients had clinically definite, relapsing-remitting MS. Healthy volunteers were selected from personnel of the Neuroimmunology lab. MS patients (n=62) studied longitudinally participated in the MASTER (MS Antibody Status and Therapy Evaluation with Rebif®) study, a clinical trial of IFN-β-1a. Eligible subjects for that study were patients at ages between 18 and 55 years who had relapsing-remitting MS for more than two years, with at least two relapses during the past two years, without any immunomodulatory treatment six months prior to the study, and without exacerbation 4 weeks prior to treatment initiation, and with a disability score of 0-5.5 as defined by the Expanded Disability Status Scale (EDSS) (Lünemann et al. 2001, Neurology 57: 1132-1134). All patients were treated with 44 μg IFN-β-1a (Rebif®, Serono, Unterschleifβheim, Germany) by subcutaneous injection once weekly. This dose has been shown to significantly decrease the T2 activity and burden of disease as measured by MRI and represents twice the concentration of IFN-β-1a that has been demonstrated to delay the conversation to definite MS in patients after a first episode of neurological dysfunction (The Once Weekly Interferon for MS Study Group 1999, Neurology 53: 679-686; Comi et al. 2001, Lancet 357; 1576-1582). Furthermore, this dosage was found to be biologically effective as indicated by the clear and significant enhancement of M×A gene expression in PBMC after initiation of treatment (cf FIG. 1). Clinical data on disability status (as defined by the EDSS) and relapses were obtained every time of blood drawing 3 days after IFN-β-1a application (Kurtzke 1983, Neurology 33: 1444-1452). A clinical relapse was defined as significant worsening for preexisting symptoms or appearance of new neurological deficits in the absence of fever and lasting for more than 24 hours. For the purpose of this study only patients with complete longitudinal data were included (n=62). Patients who experienced no further relapses and no deterioration in the EDSS during IFN-β treatment were defined as positive responders (n=20), whereas patients who continued to have one or more relapses were defined as non-responders (n=19). In some of these patients, clinical relapses were treated with a 3-day course of i.v. methylprednisolone at a dose of 1 g/day. However, all blood samples were obtained before treatment For the purpose of this study, positive responders (n=20) and non-responders (n=19) were randomly selected (cf Table 1a). In addition, patients (n=23) who developed neutralising antibodies (NAB) to Rebif® as determined by means of an M×A induction assay were included. The M×A concentration induced by Rebif® was determined as described (Kracke et al. 2000, Neurology 54: 193-199). Results are given as Interferon Neutralising Units (INU), i.e. the titre of serum that neutralises 10 U/ml Rebif® activity to an apparent 1 U/ml activity as determined by a standard curve. Within this group, 17 patients fulfilled the clinical criteria for treatment response. Venous blood samples were collected prior to IFN-β treatment (baseline=week 0) as well as at weeks 4, 26 and 52 under therapy (each time 3 days after IFN-β application). Patients who developed NAB during the treatment course were followed up until week 78.

In order to investigate the specificity and confirm our data, a second independent cohort consisted of n=20 relapsing-remitting MS patients (Table 1b) who were followed-up monthly in our Outpatient Department, and 4 healthy donors who donated blood at the same intervals. Of these, 11 patients were started on Rebif® with the standard dosage of 22 ug given 3 times per week. To alleviate side effects present at start therapy such as flu-like symptoms, the dosage was gradually increased and the full dose was achieved after-4 weeks. MRI scans (1.5 T scanner; Siemens Vision, Siemens, Erlangen, Germany) were analysed by an experienced investigator who was blinded to the clinical as well as immunological data. We determined spin echo T1—(TR 840 ms; TE 14 ms; matrix size 192×256; field of view 256 mm; 28 contiguous axial slices of 5 mm thickness), with and without contrast agent (20 ml Magnevist®, Gadopentetate dimeglumine [Gd], Schering, Berlin, Germany), and turbo spin echo T2—weighted (TR 400 ms, TE 100 ms; matrix size 96×128 mm; field of view 240 mm) scans before and 9 months after initiation of treatment. Patients who experienced neither further relapses, deterioration in the EDSS, progression in T1 and T2 lesion load, nor Gd-enhancing lesions during IFN-β treatment were defined as ‘first year responders’, whereas patients who continued to have one or more relapses or an active MRI were defined as ‘first year non-responders’.

PBMC were isolated from fresh blood by Ficoll density gradient centrifugation (Bio-Whittaker, Walkersville, Md.) and cryopreserved in liquid nitrogen. Serum was stored at −20° C. until assays were performed. All RNA and protein measurements were performed by independent investigators who were blinded to the clinical data, to the MRI data, as well as to their respective results. All parts of the study were approved by the local ethical committee, and written informed consent was obtained from each individual.

Demographical data are described by means, medians, and ranges. For reasons of comparability results in figures are expressed as mean value±SEM. Changes in interesting clinical outcomes with respect to time were analyzed using nonparametric multivariate analysis of variance (MANOVA) for repeated measurements in a two-factorial design (treatment and repetitions as factors) (Brunner, E., Domhof, S., Langer, F. Nonparametric Analysis of Longitudinal Data in Factorial Experiments. J. Wiley, New York 2002). Therefore, we compared all the points simultaneously on the corresponding response curves. After overall testing, we carried out post-hoc analyses (Wilcoxon tests) to detect specific differences (of clinical interest) between certain times of the treatment, and between treatment and baseline. For the same reason, we also analyzed differences between the two treatment groups with respect to certain time points by using the Mann-Whitney-U test. Kruskal-Wallis test was used to detect differences between more than two groups (nonparametric one-way ANOVA). Adjustments for multiple comparisons were carried out using a sequentially rejective test procedure according to Bonferroni-Hohm. Predictive discriminating values were calculated by ROC analysis. Proposals are made for optimal cut-off values taking sensitivity and specificity into consideration. Statistical analyses were performed using SPSS for Windows (Release 11.0.1, Copyright® SPSS Inc. 1998-2001) and Statistical Analysis System (SAS) version 8.2 (SAS Institute Inc. Cary, N.C.). Significance was assessed at the (exact) p<0.05 level, two-sided.

### Example 1

### Induction of TRAIL Gene Expression Upon Systemic IFN-β Treatment

First, it was examined whether systemic IFN-β treatment of MS patients induces TRAIL gene expression in vivo. For this purpose, PBMC from MS patients (n=62) before and during IFN-β-1a therapy were investigated.

Samples of peripheral blood was obtained before the beginning of IFN-β-1a therapy and at weeks 4, 26 and 52 under therapy. PBMC were isolated from fresh blood by Ficoll density gradient centrifugation (Bio-Whittaker, Walkersville, Md.) and cryopreserved in liquid nitrogen until analysis was done.

Total RNA was extracted and relative mRNA levels were quantified by realtime (rt)-PCR. In addition, the closely related TNF family member CD95L (also referred to as FasL) was quantified. In parallel, M×A protein gene expression was measured as an in vivo response marker to IFN-β-1a therapy (Wandinger et al. 2001, Ann Neurol 50: 349-357).

Isolation of total RNA from cells was carried out using the RNeasy Kit (Qiagen, Santa Clarita, Calif.), reverse transcribed to cDNA with random hexamers using the TaqMan® Reverse Transcription Reagents as per manufacturer's instructions (Perkin Elmer, Foster City, Calif.). Quantitative rtPCR was performed on an ABI Prism® 7700 Sequence Detection System (Perkin Elmer) (Wandinger et al. 2001, Ann Neurol 50: 349-357). Amplification of hypoxanthine phosphoryltransferase (HPRT) and 18S rRNA for stimulated conditions was used for sample normalisation. The amplification protocol followed the suggestions of the TaqMan® Gold RT-PCR kit. For detection of TRAIL, CD95L, M×A and HPRT transcripts, oligonucleotides were used at final concentrations of 200 nM for forward and reverse primer and 100 nM for the fluorogenic probe as follows:

| | |
|---|---|
| TRAIL forward: | 5′CAGAGGAAGAAGCAACACATTCTCT3′, |
| reverse: | 5′TGATGATTCCCAGGAGTTTATTTTG3′, |
| probe: | 5′FAM-ACTCCAAGAATGAAAAGGCTCTGGGCC- |
| | TAMRA3′; |
| CD95L forward: | 5′ATGCACACAGCATCATCTTTGG3′, |
| reverse: | 5′ATGGGCCACTTTCCTCAGCT3′, |
| probe: | 5′FAM-AAGCAAATAGGCCACCCCAGTCCACC- |
| | TAMRA3′; |
| MxA forward: | 5′CAGCACCTGATGGCCTATCAC3′, |
| reverse: | 5′GAGCATGAAGAACTGGATGATCAA3′, |
| probe: | 5′FAM-AGCAAGCGCATCTCCAGCCACATC- |
| | TAMRA3′; |
| HPRT forward: | 5′AGTCTGGCTTATATCCAACACTTCG3′, |
| reverse: | 5′GACTTTGCTTTCCTTGGTCAGG3′, |
| probe: | 5′FAM-TTTCACCAGCAAGCTTGCGACCTTGA- |
| | TAMRA3′. |

Quantification of gene expression relative to HPRT or 18S rRNA was calculated by the protocol's ΔΔC_{T} method. 18S rRNA was amplified using TaqMan® Ribosomal RNA Control Reagents (Perkin Elmer). Changes in gene expression of twofold ore more were considered valid for further statistical analysis.

Statistical analysis was performed using SPSS software (SPSS Software GmbH, Munich, Germany). The Mann-Whitney-U test was used to compare data between patient groups. For analysis of dependent variables within groups, Wilcoxon's matched pair signed-rank test was performed. Data obtained from experiments outlined below were evaluated using the same statistical approach. Demographical data are described by means, medians, and ranges. For reasons of comparability results in figures are expressed as mean value±SEM.

In FIG. 1 it is shown that, compared to baseline, spontaneous gene expression of both M×A and TRAIL was significantly up-regulated in PBMC of MS patients after 3 weeks of treatment and remained elevated for a period of 1 year (time influence proved by one-group MANOVA, each (p<0.05), post-hoc Wilcoxon tests with all (exact) p-values<0.001-α/3=0.0167 according to Bonferroni-Hohm) (FIG. 1). In contrast, CD95L did not show any significant regulation during IFN-β therapy at each time-point tested.

### Example 2

### TRAIL Gene Expression in IFN-β Responders and Non-Responders

In order to examine the functional relevance of TRAIL induction for the mechanism of action of IFN-β, patients were characterised with regard to their clinical response status. Patients negative for NAB who experienced no further relapses and no deterioration in the EDSS during IFN-β treatment were defined as positive drug responders, whereas patients who continued to have one or more relapses were defined as non-responders.

As illustrated in FIG. 2, IFN-β therapy resulted in early and sustained up-regulation of TRAIL gene expression in MS patients with clinical response (Wilcoxon tests: p=0.001, p=0.021, p=0,002 exact—Bonferroni-Hohm). Conversely, TRAIL gene expression was only transiently induced after 6 months of treatment in drug non-responders. The biological response marker M×A was significantly up-regulated in both groups (FIG. 2A), indicating the in vivo biological effectiveness of IFN-β-1a in both patient groups. As outlined in FIG. 2A, non-responders showed even higher levels of M×A gene expression in response to systemic IFN-β treatment at each time point investigated (non-parametric MANOVA p<0.0001). As for the whole group, CD95L was not significantly regulated by IFN-β therapy in the patient subsets. No differences in baseline expression levels of the genes investigated were observed between responders and non-responders (data not shown).

### Example 3

### TRAIL Gene Expression in Patients Developing Neutralising Antibodies

Given the parallel up-regulation of M×A and TRAIL expression by IFN-β in vivo, it was investigated whether the occurrence of NAB in the course of IFN-β treatment influences TRAIL gene expression as reported for M×A (Deisenhammer et al. 1999, Neurology 52: 1239-1243). Therefore, spontaneous gene expression was analysed in a third subset of patients who developed NAB after 1 year (n=23). Presence of NAB was confirmed after 18 months (cf Table 1).

FIG. 3 demonstrates that in these subjects, bioavailability of IFN-β as measured by M×A gene induction was completely inhibited in the presence of NAB (systematic time effect analysed with non-parametric MANOVA for one group p<0.0001, Wilcoxon tests with first two p-values<0.0001, p₃=0.563, p₄=0.741 exact). In parallel, initial up-regulation of TRAIL gene expression by IFN-β was abrogated, indicating a direct regulation of TRAIL transcription by IFN-β (FIG. 3) (non-parametric MANOVA for one group p<0.0001, Wilcoxon tests with first two p-values<0.0001, p₃=0.855, p₄=0.543 exact). The expression of CD95L did not show significant variations in the presence of NAB (data not shown). No significant differences in baseline gene expression levels of TRAIL were observed between the three patient subgroups (FIG. 4) (Kruskal-Wallis test, p=0.502 exact).

Interestingly, the majority of these patients remained in the clinical response status in the study time. However, this clinical observation is in keeping with the results of the pivotal trial of IFN-β-1b and the extension of the PRISMS study of IFN-β-1a, where development of NAB was finally associated with a delayed loss of efficacy in an observation period between 18 and 24 months and after 24 months, respectively (The IFNB Multiple Sclerosis Group and the University of British Columbia MS/MRI Analysis Group 1996, Neurology 47: 889-894; The PRISMS Study Group and The University of British Columbia MS/MRI Analysis Group. PRISMS-4, 2001, Neurology 56: 1628-1636).

### Example 4

### Protein Expression of TRAIL in IFN-β Responders and Non-Responders

To address the question whether IFN-β non-responders differed from responders also with regard to TRAIL protein expression, serum concentrations of sTRAIL protein before and during IFN-β-1a therapy was measured in two different experimental settings.

For the experiments, serum of NAB negative drug responders (n=12) and non-responders (n=11), from whom serum was available for investigation, was stored at −20° C. until the assay were performed. Soluble TRAIL protein was quantified using a sandwich ELISA with a detection limit of 65 pg/ml according to the manufacturer's protocol (Trinova Biochem, Gieβen, Germany).

The inventors measured membrane bound and soluble TRAIL (sTRAIL) protein levels before and during IFN-β-1a therapy on PBMC and in serum of NAB negative drug responders (n=12) and non-responders (n=11) in whom serum was available for investigation. Before therapy, MS patients (n=23) revealed lower sTRAIL levels compared to sex and age-matched healthy individuals (n=24) (Mann-Whitney-U test p=0.017 exact) (FIG. 5A). Within the patient group, there were significant differences in sTRAIL protein accumulation in the serum between responders and non-responders in the course of IFN-β treatment (MANOVA, p=0.00109) (FIG. 5B).

Soluble TRAIL protein levels were measured in serum of IFN-β responders (n=12) and non-responders (n=11) before the beginning of IFN-β-1a therapy and at weeks 4, 26 and 52 under therapy. Box-whisker plots depict the median level for each group (horizontal line in the box), the 25^{th} and 75^{th} percentiles (upper and lower edges of box), and the 10^{th} and 90^{th} percentiles (lines extending above and below the box). Statistical significance of differences between the patient group was tested by the Mann-Whitney-U test. *p<0.05.

As demonstrated in FIG. 5B, responders showed significantly higher sTRAIL baseline levels compared to non-responders already prior to the beginning of treatment (Mann-Whitney test p=0.004 exact), indicating underlying differences in the post-transcriptional regulation of TRAIL expression. Levels of sTRAIL above 350 pg/ml, in particular at approximately 584.1 pg/ml were definitely associated with response to IFN-β therapy. Thus, levels of sTRAIL above or slightly below 350 pg/ml prior to therapy predicted the clinical outcome in 92% of the positive responders and 73% of the non-responders, respectively. For the total group of patients, this implied a correct prediction rate of 83.6%. Compared to baseline levels, sTRAIL protein accumulation in the serum was significantly enhanced after 4 weeks of IFN-β treatment in responders and non-responders. In long-term treated patients, sTRAIL protein levels remained significantly up-regulated in positive responders compared to non-responders after 6 and 12 months (Mann-Whitney test: p(6 months)=0.011, p(12 months)=0.015 exact—Bonferroni-Holm).

In addition to sTRAIL in blood serum, membrane bound TRAIL protein expressed on PBMC was measured using flow cytometry. PBMC from NAB negative responders and non-responders before and during IFN-β-1a therapy were prepared and 2×10⁵ PBMC per sample were incubated with an anti-TRAIL monoclonal antibody (cat. no. 804-322-C100, Alexis Corporation, San Diego, Calif.) or control mouse IgG1 (cat. no. X0931, DAKO, Glostrup, Denmark) at a concentration of 1:100 for 30 min at 4° C. Washed cells were incubated with a phycoerythrin (PE)-labelled secondary goat-anti-mouse antibody (cat. no. R0480, DAKO) at a concentration of 1:20 for 30 min. After washing with phosphate buffered saline (PBS), cells were analysed on a FACScan flow cytometer and data were processed using the Cellquest software (Becton-Dickinson, Mountain View, USA). Values are expressed as specific fluorescence intensity, calculated by histogram statistics and defined by the percentage of fluorescence intensity with TRAIL antibody excluding the fluorescence intensity with the appropriate control antibody.

Significant changes in TRAIL surface expression on PBMC during IFN-β treatment in any of the patient subsets were not observed (data not shown). Since blood samples were collected from patients 3 days after IFN-β application (subcutaneous injection) throughout the study, the negative findings might be explained by the time course of membrane bound TRAIL expression.

### Example 5

### TRAIL Gene and Protein Expression in PBMC Cell Culture

PBMC were cultured in the presence of IFN-β-1a in vitro and the regulation of TRAIL gene transcription, cell surface expression and accumulation of soluble protein in culture supernates were investigated in comparison (cf FIG. 6).

Freshly thawed PBMC were resuspended in Iscove's modified Dulbecco's medium (Gibco, Grand Island, N.Y.) supplemented with 2 mM L-glutamine, 50 mg/ml gentamycin and 100 U/ml penicillin/steptomycin (Whittaker Bioproducts, Gaithersburg, Md.) and 5% human plasma. PBMC (10⁶ cells per well) were incubated in a humidified atmosphere of 5% CO₂ at 37° C. Where specified, phytohaemagglutinin (PHA) or cytokines were added to the cultures as follows: PHA 1 μg/ml (Sigma-Aldrich, Steinheim, Germany), recombinant human IFN-β-1a, 10 and 100 IU/ml (Rebif®, Serono, Unterschleissheim, Germany), recombinant human IL-2, 10 IU/ml (Proleukin, Eurocetus, Frankfurt, Germany).

As demonstrated in FIG. 6A, incubation of PBMC with IFN-β resulted in rapid, dose-dependent up-regulation of TRAIL mRNA levels after 24 hrs, followed by a second peak after 72 hrs. TRAIL expression was likewise rapidly induced on the surface of PBMC after 24 hrs by IFN-β (FIG. 6B). However, expression of membrane bound TRAIL was only marginally enhanced compared to baseline levels after 72 hrs. Soluble TRAIL concentrations in culture supernates increased over 24 hrs and remained elevated for 96 hrs when stimulated with IFN-β at a concentration of 10 IU/ml (FIG. 6C). Incubation with IFN-β at a concentration of 100 IU/ml resulted in further increase of sTRAIL over 96 hrs. In contrast to IFN-β, stimulation of cells with PHA and IL-2 resulted in delayed TRAIL gene and protein expression (FIG. 6A-C). Consistent with previously reported findings, these data indicate that IFN-β directly regulates the transcriptional control of TRAIL expression (Kayagaki et al. 1999, J Exp Med 189:1451-1460).

### Example 6

### Confirmation of Differential TRAIL Regulation in Patients Treated with a Higher IFN-β Dose

In order to test the hypothesis of TRAIL as a potential response marker for IFN-β, further TRAIL expression was studied in a second, independent patient group that was started on IFN-β therapy 22 μg three times weekly. In these patients, treatment response was confirmed by MRI analysis as a measure of subclinical disease activity. In addition to clinical criteria, responders revealed no progression in T1 and T2 lesion count and lacked new Gd-enhancing lesions during therapy. As a confirmation of the findings, responders could clearly be discriminated from non-responders during treatment by significant differences in TRAIL gene induction (sys\acute{t}ematic time effect analysed by non-parametric MANOVA p=0.026) (FIG. 7A).

The impact of TRAIL gene induction by IFN-β given in the standard dosage was even almost 5 fold higher in these patients, demonstrating dose-dependent regulation of gene transcription also in vivo. The biological response marker M×A was significantly upregulated in responders and non-responders (systematic time effect analysed with non-parametric MANOVA for one group each p=0.0064 and p=0.0006, resp., exact) (FIG. 7B).

The comparison of the IFN-β treated patients with a group of 9 untreated patients and 4 healthy individuals underlines the specificity of TRAIL upregulation in patients responsive to IFN-β therapy (FIG. 7A). Neither in the group of untreated patients nor in the healthy individuals M×A was increased (FIG. 7B).

### Example 7

### Predictive Value of sTRAIL for IFN-β Therapy Response

Since also the second group of IFN-β treated patients exhibited elevated sTRAIL levels in the responders prior to treatment, finally an overall ROC-analysis in order to determine the predictive discriminating value of sTRAIL for therapy response was performed. Altogether, 49 patients prior to treatment (including NAB+ patients) from whome sera were available were investigated, subsequently comprising 29 responders and 20 non-responders. Levels of sTRAIL above or below 584.1 pg/ml prior to therapy correctly predicted the clinical outcome in 90.5% of the responders and 71.4% of the non-responders. The characteristics of the ROC analysis (area under the curve: 0.879, 95% confidence-interval: 0.785-0.974) are given in FIG. 8A. To investigate whether patients with neutralising antibodies (NAB) had a bearing on the ability of sTRAIL to predict treatment response, a sensitivity analysis in form of a second prediction analysis without the NAB patients was performed (FIG. 8B). Since the results of this subgroup (area under the curve: 0.919, 95% confidence-interval: 0.812-1.026) were in accordance with the analysis in all patients, one can assume that these patients have no relevant impact on the capability of sTRAIL as a prognostic marker.

The features disclosed in the foregoing description, in the claims and/or in the accompanying drawings may, both separately and in any combination thereof, be material for realising the invention in diverse forms thereof.

1-21. (canceled)

22. A method for identifying an individual as a positive responder or a non-responder to a type-1 Interferon (IFN) therapy used in the treatment of Multiple Sclerosis, wherein said method comprises determining TRAIL expression and/or TRAIL protein level and correlating the determined TRAIL expression and/or TRAIL protein level with responder or non-responder status.

23. The method according to claim 22, wherein the therapy used in an IFN-β therapy.

24. The method according to claim 22, wherein TRAIL expression determination and/or TRAIL protein level determination is done prior to IFN therapy.

25. The method according to claim 22, wherein TRAIL expression determination and/or TRAIL protein level determination is carried out with a sample of mammalian origin.

26. The method according to claim 25, wherein the mammal is a human.

27. The method according to claim 26, wherein the sample is selected from the group consisting of bodily fluids, or a fraction thereof; tissue extracts; and cell extracts.

28. The method according to claim 27, wherein the bodily fluid is selected from the group consisting of whole blood or a fraction thereof; blood serum; plasma; cerebrospinal fluid; synovial fluid; ascites exudates; inflammatory exudates; and urine.

29. The method according to claim 22, wherein
(a) TRAIL expression is determined by detecting and/or measuring the amount and/or concentration of TRAIL nucleic acids encoding the TRAIL polypeptide or a part thereof, and
(b) TRAIL protein level is determined by detecting and/or measuring the amount and/or concentration of TRAIL polypeptide or a part thereof.

30. The method according to claim 29, wherein the TRAIL protein level determination is performed by measuring the amount and/or concentration of soluble TRAIL protein in a liquid sample.

31. The method according to claim 29, wherein the measurement of the amount and/or concentration of TRAIL polypeptide is done by an immunoassay using an anti-TRAIL antibody.

32. The method according to claim 29, wherein the amount and/or concentration of TRAIL polypeptide determined is compared to a limiting value discriminating positive responders and non-responders.

33. The method according to claim 32, wherein the limiting value is at least about 350 pg/ml.

34. The method, according to claim 33, wherein the limiting value is about 584 pg/ml.

35. The method according to claim 29, wherein the amount and/or concentration of TRAIL nucleic acids is determined by measuring the amount and/or concentration of TRAIL mRNA in a sample.

36. The method according to claim 35, wherein the sample is selected from the group consisting of extracts of blood cells; peripheral immune cells; neuronal tissue; and muscle tissue.

37. The method according to claim 34, wherein measurement of the amount and/or concentration of TRAIL mRNA is performed with the aid of the polymerase chain reaction using one or more TRAIL specific probes.

38. The method according to claim 35, wherein the amount and/or concentration of TRAIL mRNA is compared to a limiting value discriminating positive responders and non-responders.

39. The method according to claim 22, wherein the amount and/or concentration of TRAIL nucleic acids is correlated with the amount and/or concentration of nucleic acids of one or more biological activity marker(s), and/or the amount and/or concentration of TRAIL polypeptide is correlated with the polypeptide amount and/or concentration of one or more biological activity marker(s).

40. The method according to claim 38, wherein the biological activity marker is an IFN-inducible protein.

41. The method according to claim 39, wherein the biological activity marker is an M×A protein.

42. A method for monitoring IFN therapy used in the treatment of Multiple Sclerosis wherein said method comprises determining TRAIL expression and/or TRAIL protein level and correlating the determined TRAIL expression and/or TRAIL protein level with the progression of treatment.

43. A kit for identifying an individual as a positive responder or a non-responder to a type-I Interferon (IFN) therapy used in the treatment of Multiple Sclerosis or for monitoring IFN therapy used in the treatment of Multiple Sclerosis, wherein said kit comprises a nucleic acid probe and/or primer(s) specific for the TRAIL gene or a portion thereof and/or an antibody specific for the TRAIL polypeptide or a portion thereof.

44. The kit according to claim 43, comprising at least one antibody specific for the TRAIL polypeptide that is suitable for an ELISA assays.
